(19) United States
(12) Patent Application Publication
(10) Pub. No.: US 2005/0277841 A1
(43) Pub. Date: Dec. 15, 2005

(54) Disposable fetal monitor patch

(76) Inventor: Adnan Shennib, Dublin, CA (US)

Correspondence Address: GLENN PATENT GROUP, 3475 EDISON WAY, SUITE L, MENLO PARK, CA, 94025, US

(21) Appl. No.: 10/866,378

(22) Filed: Jun. 10, 2004

(51) Int. Cl. A61B 5/0444

(52) U.S. Cl. 600/511

(57) ABSTRACT

The invention provides a low cost, fully integrated, disposable patch for the non-invasive, continuous monitoring of fetal electrocardiogram (ECG). The patch detects fetal ECG by filtering the dominant maternal ECG therefrom. In one embodiment, an upper electrode is used to obtain a relatively pure maternal ECG signal for its cancellation from the signal obtained from the abdominal fetal ECG. In another embodiment, multiple abdominal electrodes are used and the dominant periodic features of maternal ECG are identified and eliminated. The fetal monitor patch is thin, flexible, and incorporates a battery and an alarm within. The alarm is activated during an adverse health condition for the fetus. The fetal monitor patch is particularly designed for long-term wear applications exceeding one week and lasting up to several months. The patch is unobtrusive and thus worn continuously, even during sleep and bathing. In another embodiment, the fetal monitor patch is programmable and stored fetal ECG data can be transmitted to a remote receiver.

### BACKGROUND OF THE INVENTION

1. Technical Field

The invention relates to non-invasive monitoring of fetal vital signs. More particularly, the invention relates to fetal electrocardiogram (ECG) monitoring.

2. Description of the Prior Art

Techniques to monitor the fetal status during pregnancy have been developed and are widely used in clinical settings. These methods are necessary to detect possible abnormalities. Early detection of fetal morbidity can have a profound influence on the fetal outcome.

Monitoring of fetal heart activity is particularly useful in assessing the general health of the baby, as well as the baby's vascular system in particular. Vital signs, such as fetal heart rate and beat-to-beat rate, and variability are altered by the sympathetic and parasympathetic nervous system, and thus provide an excellent indication of the well-being of the baby. For example, the absence of variability in fetal heart rate is an ominous sign requiring further investigation and possible intervention by medical personnel.

The high cost and inconvenience of current instruments excludes continuous long term monitoring of high-risk pregnancies. This effectively eliminates the possibility of detecting abnormalities in normal and low risk pregnancies. Monitoring of fetal heart rate can half the incidence of neonatal seizures, which have a close correlation with long-term handicaps (Kam, 1999). In addition to the diagnosis of the general well-being of a fetus, fetal ECG monitoring is particularly useful in detecting congenital heart abnormalities which are present in approximately 0.5-0.8% of all deliveries in the normal population.

There are several methods commonly used today in non-invasive fetal monitoring:
acoustic, ultrasonic, and electrocardiography (ECG).

Acoustic methods involve obtaining fetal acoustics, including heart sounds. This includes using a fetoscope, a stethoscope, or phonographic instruments employing acoustic transducers. However, acoustic fetal monitors are generally difficult to administer, particularly for self-administration, require training, and generally provide limited diagnostic data.

Ultrasonic methods use reflected acoustic energy in the ultrasonic range to localize and visualize various fetal structures, including heart valves. Heart rates can also be detected using ultrasonic instruments. However, ultrasonic monitoring requires training and the results lack electrophysiologic information. It also requires proper alignment, and thus can be a challenge for self-administration when considering the movement of the fetus in the uterus. Ultrasonic equipment is expensive and consumes a large amount of power, and thus is not suitable for long-term battery-operated applications. For the above reasons, ultrasound monitoring has not been widely employed in ambulatory applications, particularly at home settings.

Fetal ECG monitoring provides essential diagnostic data particularly that pertaining to the heart. Invasive methods involve placing an electrode on the scalp of the fetus during delivery time. Other invasive methods involve inserting an electrode inside the uterus, i.e. U.S. Pat. No. 5,431,171 to Harrison et al, and U.S. Pat. No. 6,115,624 to Lewis et al Obviously, invasive methods are not practical for screening and ambulatory applications because they generally require the rupture of the protective amniotic sac.

Body surface potential of ECG from the mother's abdomen is non-invasive but has many challenges. First, the fetal ECG signal is highly contaminated with the maternal ECG, which may be an order of magnitude stronger than the fetal ECG signals. Second, the fetal ECG signal is inherently weak, and thus easily contaminated by electromagnetic interference (EMI) from power lines and equipment, as well as electromyogram (EMG) from muscle activity.

FIG. 8a is a waveform for a typical fetal ECG with both fetal and mother ECG features shown. The QRS complex of the fetus (QRS_{f}) is typically weak as compared to the dominant mother QRS (QRSₘ). Other ECG features of maternal ECG can also be seen, including the T-wave (Tₘ). There is no place on the mother's skin whereby only the fetal ECG can be obtained. However, the ratio of fetal ECG to maternal ECG can be improved substantially when measuring ECG at the abdomen area. Regardless of the strength of fetal ECG, additional processing is necessary to extract fetal ECG and its features for the purpose of identifying cardiac parameters such as average fetal heart rate and beat-to-beat rate.

Several signal processing algorithms and methods are widely used in relatively large computer-based systems for fetal ECG filtering, including the least mean square (LMS) method, Recursive Least Square (RLS), Blind Source Separation (BSS), Genetic Algorithms, and fuzzy logic. Furthermore, combinations of signal processing methods have been applied for the proper filtering and detection of fetal ECG features. However, even with advances in instrumentation and signal processing methods, current proposed systems are generally bulky and limit the monitoring to clinical setups in the presence of trained personnel. For example, see U.S. Pat. No. 5,123,420 to Paret, U.S. Pat. No. 5,372,139 to Holls et al, and U.S. Pat. No. 5,042,499 to Frank et al. These prior art instruments and methods are expensive and exclude home monitoring and are typically limited to high-risk pregnancies.

U.S. Pat. No. 4,781,200 to Baker discloses a system for automatic and continuous monitoring the well-being of a fetus. Baker's device incorporates a belt garment with multiple sensors worn about the mother's abdomen. The device incorporates a control unit 40 (FIG. 1 of Baker) attached to the belt garment. The control box incorporates a display, an alarm, and means for processing multiple physiologic parameters, and is particularly suited for indicating movements of the fetus. Although less bulky and more suited for ambulatory purposes than prior art mentioned above, Baker's invention is relatively complex, expensive, and cumbersome for expectant mothers, particularly during sleep when considering the physical profile of the control box.

One object of the invention is to provide a fetal monitor device and method that is unobtrusive and that can be worn continuously and conveniently by an expectant mother at home.

A further objective of the invention is to provide a low cost fetal monitor that is suitable for use by all pregnant mothers, including those with normal and low risk pregnancies.

A further objective is to develop an automated fetal monitor, which eliminates supervision or intervention by medical personnel.

A further objective is to provide real-time fetal heart indications, particularly an alarm during adverse conditions.

### SUMMARY OF THE INVENTION

The invention provides a low cost patch for the non-invasive monitoring of a fetus. The patch is adhered on the abdomen area of an expectant mother for continuous and automatic monitoring of fetal electrocardiogram (ECG). The fully integrated monitor patch detects the surface potentials present on the abdomen area and filters out the maternal component of ECG which contaminates fetal ECG. Filtering is accomplished by a combination of proper electrode placement and signal processing. In one embodiment, an upper electrode obtains a relatively pure maternal ECG signal that is used for the cancellation of maternal ECG component from the abdominal fetal ECG. In another embodiment, the dominant periodic features of maternal ECG are identified and eliminated from measurements obtained from multiple abdominal electrodes.

The fetal monitor patch is thin, flexible, and incorporates a battery and an alarm within. The alarm is activated during an adverse health condition for the fetus. In the preferred embodiment, the fetal monitor patch is disposable, and is thus discarded upon battery depletion. Although particularly useful for monitoring high-risk pregnancies, the simplicity and low cost aspect of the invented patch allow for use by all pregnant women.

The fetal monitor patch is particularly suited for long-term wear exceeding one week and lasting up to several months. The patch is worn continuously even during sleep and showering, and is thus made durable and waterproof, while being flexible and unobtrusive, for inconspicuous wear underneath clothing. Alternatively, the fetal monitor patch can be used for short term or spot check applications.

Real-time fetal heart activity can be indicated to the mother for continuous assurance of fetal health. This is accomplished by providing an audible tone or a flashing signal in sync with fetal QRS events.

In another embodiment for diagnostic applications, the fetal monitor patch is wirelessly programmable using an external programmer. The programmable patch collects fetal ECG data in memory while providing a real-time monitoring and indications for the pregnant mother. The fetal ECG data is then transmitted to a clinic via a telephone, a personal computer connected to the Internet, or by an interrogation device at the clinic.

### BRIEF DESCRIPTION OF THE DRAWINGS

FIG. 1 is a frontal view of a fetal monitor patch placed on the abdomen of an expectant mother, in which the patch is vertically elongated with an upper electrode for cancellation of maternal ECG component;

FIG. 2 is detailed view of the vertically elongated fetal monitor patch of FIG. 1 showing the major internal components;

FIG. 3 is a cross section view of the fetal monitor patch in FIG. 2;

FIG. 4 is a detailed cross section view of a section of the fetal monitor patch of FIG. 2, showing the various layers including a metal foil layer;

FIG. 5 shows a rectangular embodiment of a fetal monitor patch having three electrodes;

FIG. 6 shows a 5-electrode embodiment placed on the abdomen of an expectant mother;

FIG. 7 is a schematic diagram of the electronic assembly within the fetal monitor patch, showing audible and visual indicators and wireless control by an external magnet;

FIG. 8a shows the fetal ECG contaminated by the dominant maternal ECG;

FIG. 8b shows extract QRS complex of the fetal ECG;

FIG. 9 is a block diagram of a typical signal processing algorithm and a multiplexer for electrode selection;

FIG. 10 shows an embodiment of the fetal monitor patch having two maternal ECG electrodes;

FIG. 11 shows an abdominal-only electrode configuration of the fetal monitor patch;

FIG. 12 shows a block diagram of adaptive filtering of ECG signals from an abdominal-only fetal monitor patch;

FIG. 13 shows a fetal monitor patch placed on the side of the abdomen;

FIG. 14 shows a programmable fetal monitor patch having a wireless programming device with a programming coil in proximity to a wireless sensor incorporated in the patch; and

FIG. 15 shows a fetal monitor patch equipped with acoustic transducers for transferring ECG data acoustically over the telephone.

### DETAILED DESCRIPTION OF THE INVENTION

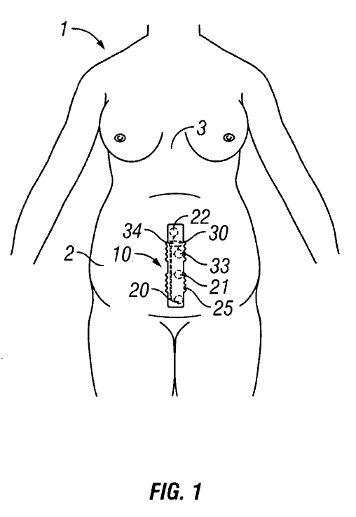

The invention, shown in various embodiments of FIGS. 1-7, 10, 11 and 13-15, is non-invasive fetal electrocardiogram (ECG) monitoring device 10 in the form of a patch placed on the abdomen area 2 of an expectant mother 1. The patch device 10 is thin and flexible for unobtrusive continuous wear.

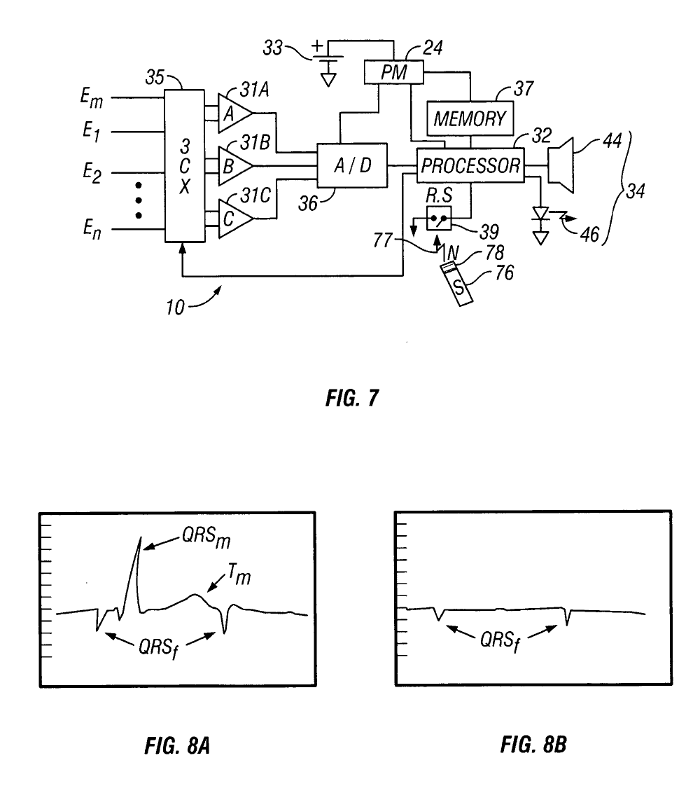

Referring to the embodiment of FIGS. 1-3, the patch device 10 comprises a lower abdomen electrode 20 for obtaining fetal ECG signal, a reference electrode 21, and a maternal electrode 22 for obtaining relatively pure maternal ECG. The device 10 comprises an electronic assembly 30 including an ECG amplifier 31, a processor 32, and a power source 33. The processor 32 is typically a digital signal processor for performing numerical computation from data obtained from an analog to digital converter 36 (FIG. 7).

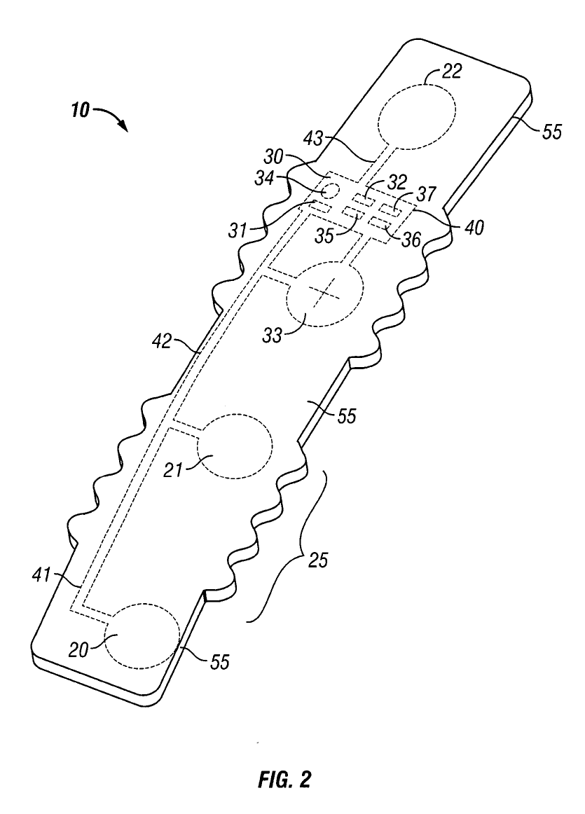

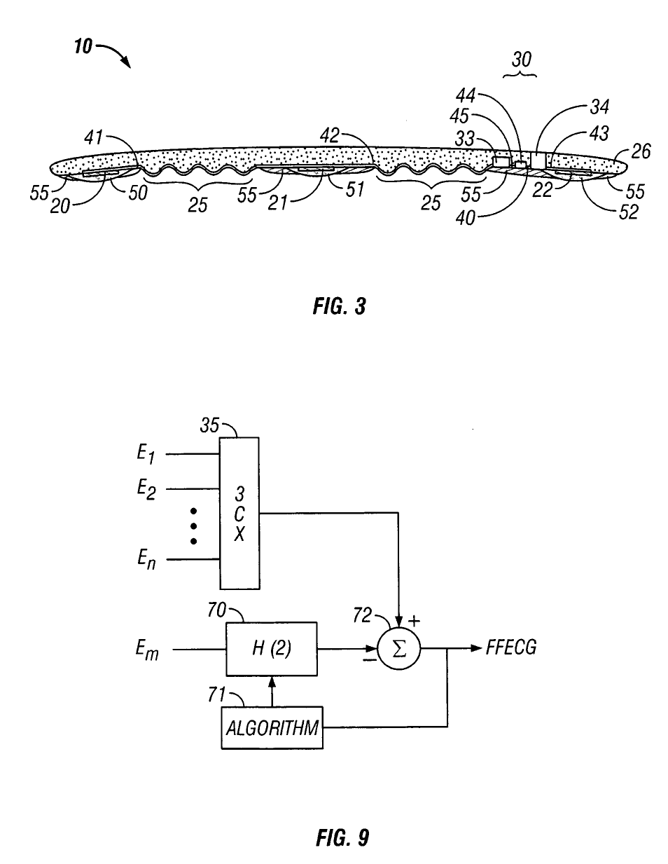

In a more detailed view of the device shown in FIGS. 2-4, the electronic assembly 30 is mounted on a flexible circuit substrate 40 with trace extensions 41, 42, 43 and 45 connecting the electronic assembly 30 to electrodes 20, 21, 22 and the power source 33, respectively. Conductive adhesive films 50, 51 and 52 cover metal electrodes 20, 21 and 22, respectively. Conductive adhesive films 50, 51, and 52 contact the skin directly to conduct surface ECG potentials to the ECG amplifier 31. A non-conductive adhesive 55 provides an overall adhesive to secure the patch device 10 to the body. The device 10 also comprises a thin substrate 26 (FIG. 3-5) for providing structural support. The substrate 26 is made of soft flexible sheath material, such as polyurethane or cloth. The thickness of the patch device 10 is preferably in the range of 1.5 and 2.5 mm but no more than 3.5 mm.

The patch assembly 10 may comprise as few as two electrodes or as many as five or more electrodes, depending on the desired fetal ECG results. Two or three electrodes are sufficient for basic monitoring applications, whereby only the basic features (also known as singular points) of fetal ECG are required, such as for the identification of R-wave. In these embodiments, feature extraction of maternal and fetal ECG based on singular value decomposition is applicable. Feature extraction of fetal R-wave is particularly useful due to its intensity relative to other fetal ECG waveform features.

FIG. 1-3 show an elongated patch arranged in a vertical electrode configuration. One advantage of this configuration is that it places at least one electrode near or at the chest area 3 for obtaining a relatively pure maternal ECG signal. FIG. 5 shows an alternate 3 electrode configuration whereby the patch is rectangular in shape, having a single upper electrode (E_{M}), and two electrodes, E_{R,}, E_{L} for placement on the right and left sides of the lower abdomen.

Recent research indicates that a more detailed feature extraction of fetal ECG signals can be valuable in detecting vascular abnormalities of the fetus. This type of diagnostic analysis would require additional details of fetal ECG not easily attained with two or three electrodes.

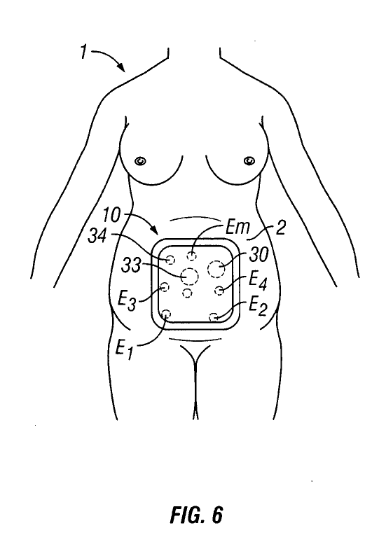

FIG. 6 shows a 5-electrode embodiment, having an upper electrode E_{M} for maternal ECG monitoring and four abdominal electrodes E₁, E₂, E₃ and E₄, for fetal ECG monitoring.

The multi-abdominal electrode configuration is also useful in applications to minimize the effects of fetal position movement in the uterus, thereby ensuring the strongest fetal ECG signal possible regardless of fetus position. This is partially accomplished by the application of a multiplexer (MUX, 35; FIG. 7), whereby any two electrode leads can be paired as a differential input to the ECG amplifiers 31A, 31B, 31C. Because the multiplexer 35 is under the control of the processor 32, network selection of electrodes can be dynamically performed in real-time for obtaining the desired fetal ECG signal.

Optimal fetal ECG signal is also partially accomplished by the application of adaptive signal processing algorithms. In its simplest form shown in FIG. 9, filtered fetal ECG is obtained by optimizing a filter function H(z) 70 by an adaptive filtering algorithm 71, leading to optimal cancellation of the maternal ECG component from the fetal ECG.

Because fetal ECG is typically an order of magnitude smaller than maternal ECG (see FIG. 8a), the optimal algorithm is obtained when filtered fetal ECG magnitude is minimized at the output of the summer 72. The optimization process is made periodically to select optimal abdominal electrode selection dynamically (FIG. 9), or pairing (FIG. 7) of electrodes E₁ through Eₙ. FIG. 8b shows filtered fetal ECG with maternal ECG components removed and fetal QRS (QRS_{f}) identified.

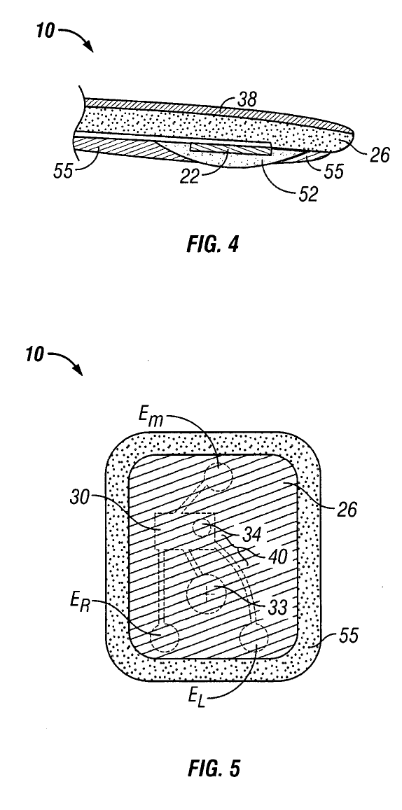

Various filtering methods are known in the field of signal processing and particularly pertaining to ECG signals. Filtering is not only necessary for removing the maternal component of ECG but also for filtering out various noise forms, such as electromagnetic interference (EMI) and muscle activity (EMG). For example, notch filters are effective in removing 60 Hz noise present in the environment. To minimize interference further, a metal foil 38 (FIG. 4) is preferably provided over the substrate 26, either over the entire device patch, or selectively over certain electronic traces and components sensitive to interference.

The power source 33 in the preferred embodiments is a primary battery with long shelf life. However, a rechargeable power source, such as rechargeable battery or charge capacitor, can be employed in conjunction with an external charging device (not shown). Wireless recharging methods are well known in the field of biomedical implants including inductive coupling whereby a coil within the device (not shown) is used to receive a charging energy from an external coil introduced in proximity.

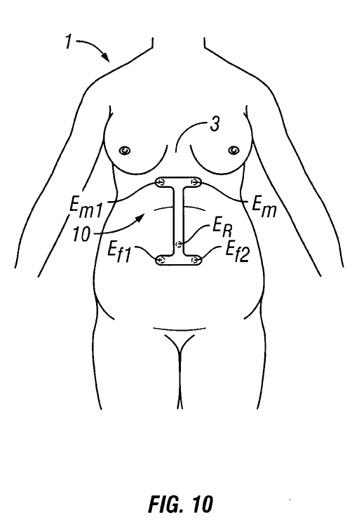

Other configurations of the invented patch include multiple maternal electrodes, as shown in FIG. 10. In this configuration, two maternal electrodes Eₘ₁ and Eₘ₂ are used for receiving relatively pure maternal ECG and two abdominal electrode E_{f1} and E_{f2} for receiving fetal ECG contaminated with maternal ECG component. A reference electrode E_{R} is used as a reference node for both maternal and abdominal measurements.

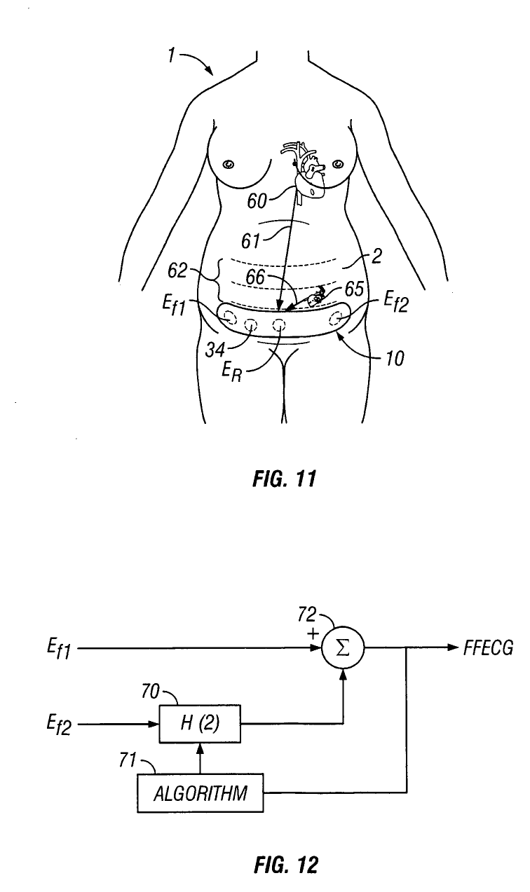

In yet another embodiment, abdominal-only electrodes are provided as shown in FIG. 11. This configuration works on the principle of equal-potential contours 62, which are orthogonal to the maternal ECG vector 61 emanating from the maternal heart 60, whereby the ECG waveform is substantially similar along a particular equal-potential contour. In contrast, the fetal ECG vector 66, emanating from the fetal heart 65, results in substantially varied waveform at points along a maternal equal-potential contour. By extracting the highly similar maternal ECG component from multiple abdominal electrodes along a maternal equal-potential contour, a filtered fetal ECG is obtained. In this particular embodiment, abdominal electrodes E_{f1}, E_{f2} and E_{R} are substantially aligned horizontally as shown in FIG. 11. To enhance the cancellation of a maternal ECG, a filtering function H(z) 70 (FIG. 9) is applied with an adaptive signal processing algorithm 71 to produce optimal cancellation signal at input of the summer 72 and resulting in a filtered fetal ECG (FFECG) at the output.

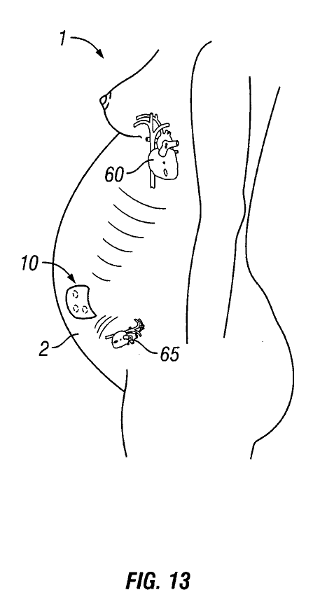

FIG. 13 shows another embodiment placing the fetal monitor patch device 10 on the side of the abdomen. Other embodiments envisioned (not shown) include providing an abdominal patch extending to the back of an expectant mother.

A major feature of the abdominal patch of the invention is the incorporation of an indicator transducer 34 for indicating the status of the fetus to the mother. For example an alarm transducer is activated during a hazard event detected by the monitor device 10. The indicator transducer 34 may be in the form of an audible transducer (44, FIG. 7), such as a buzzer or a speaker; or it may be in the form of visual display 46, such as a light emitting diode (LED) or a liquid crystal display (LCD). Another example of an indicator transducer is a vibrating element for imparting tactile sensations for the mother. The indicator may also be used to indicate other cardiac activity, such as fetal heartbeat events. For example, beeping sounds or LED flashes synchronized with fetal heartbeats detected by the patch device system.

The use of Blind Source Separation (BSS) or any other suitable algorithm may also be used to detect further and separate the ECG of twins. Multiple gestation cases (mostly twins) occur in about 1% of all pregnancies. The indication for twin ECG must be distinguished appropriately from single fetal ECG. For example, by presenting double beeps, double flashes, or alternatively presenting a different pitch or tone for each fetal ECG.

The heart activity indication through indicator transducer 34 is preferably under the remote control of the mother for activation and deactivation. For example, the mother may choose to turn off sounds representing QRS, to create a quiet mode of operation. For reassurance, these sounds can be reactivated by the mother periodically. Similarly, visual indications can also be activated and deactivated by the mother.

FIG. 7 is a schematic diagram that shows major components of an embodiment comprising a remote control device 76 in the form of magnet 78 having a magnetic field 77. A reed-switch 39 (wireless sensor) incorporated in the patch device 10 responds to the magnetic field 77 of the magnet when introduced in proximity thereto. The triggering of the reed-switch by the magnetic field (closure of the reeds) causes the sound mechanism 44 and/or visual display 46 display to toggle between activation and deactivation. However, it must be understood that heartbeat indication is separate and distinct from alarm indication, and thus both must be present in clearly differentiated forms.

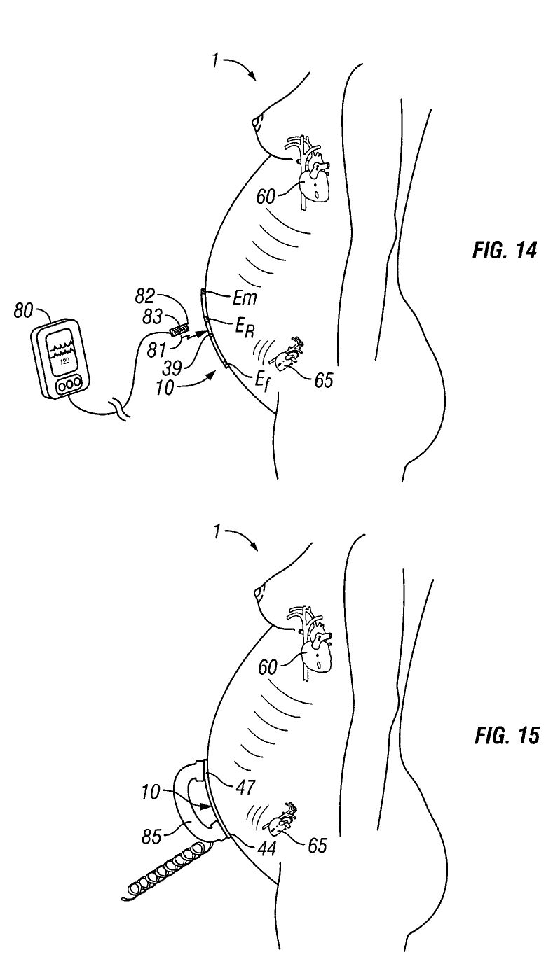

In another embodiment of the invented fetal monitor shown in FIG. 14, the device is programmable to configure the operational parameters of each patch individually according to the needs and condition of the expectant mother. Operational parameters include sampling rate, filtering algorithm, electrode position and selection, alarm indication method, i.e. alarm tone selection, and alarm indication criteria, Programming is preferably by wireless means incorporating a wireless receiver 39 to receive coded wireless commands 81 from a transmitter 82 of an external programming unit 80. In FIG. 14, the wireless receiver 39 is a miniature reed-switch for receiving magnetic pulses from an electromagnet coil 83 incorporated in the transmitter 82. The transmitter is preferably in the form of hand-held wand.

Furthermore, possible features include the ability to transmit ECG data stored in memory 37 to a remote receiver (not shown) for display and clinical analysis by a medical staff. For example, FIG. 15 shows acoustic trans-telephonic transmission of data from an audio transducer 44 incorporated within the patch device 10 to the mouthpiece of the telephone handset 85. In this embodiment, acoustic interrogation commands from the remote unit via the earpiece of the handset can also be downloaded into the patch device 10 via the receiver audio transducer 47. It should be obvious that both fetal and maternal ECG can be stored and transmitted to a remote receiver.

The wireless reception of commands and transmission of data may be accomplished in numerous ways and methods known in the field of remote control and wireless transmission of data. This includes optical, radio frequency (RF), magnetic, ultrasonic, and acoustic transmission. Furthermore, the indicator transducer 34 mentioned above can be used for the dual function of heart activity indication and data transmission. For example, a buzzer can be used to sound an alarm, as well as to send ECG data acoustically to remote location or a receiver unit in a clinical setup. Similarly, an LED indicator can be used to indicate heart activity to the mother, as well as to send ECG data to a receiver unit equipped with an optical detector. The programming unit 80 (FIG. 14) can also serve as a receiver unit. The combined programming/receiver unit can be a desktop, a portable, or a handheld instrument.

The invented fetal monitor patch is particularly designed for long-term wear by the expectant mother. For this reason, many design details are incorporated for the device to function properly and reliably for extended periods of time exceeding one week and lasting to several months. The adhesion to the abdomen skin may be designed for single-use or multiple applications. In single-use applications, the patch device is applied once for continuous wear until removed for its disposal several weeks later. In this case, the patch is worn even during sleep and bathing. In multiple applications design, the adhesive allows for multiple removal and reapplication to the skin. In either design, the adhesive 55 incorporated in the device 10 must provide continuous reliable adhesion to prevent inadvertent peeling of the device from the abdomen skin. A biocompatible skin adhesive, such as hydrogel and like materials, has been shown to be effective in human skin applications. The ideal properties of the skin adhesive include being waterproof and air-permeable. Waterproof properties aid in the protection of the electrode area underneath the patch from water-born contaminants. Air permeability properties allow for the healthy aeration of the tissue underneath patch device.

To achieve longevity of operation for the patch device, various means for power conservation must be considered. This includes power management (PM) circuitry (24 FIG. 7) to shut off certain electronic components selectively when not in use. The patch device 10 also incorporates stretchable areas 25 to allow for abdomen expansion expected during the gestation period. The construction of the device must be durable and protective of the components within. Metal foil 38 covering the internal components and substrate 26, not only provides EMI protection, but also water proofing and overall protection.

Proper patch adhesion to the skin is not only important for waterproofing purposes, but also to maintain proper electrode-skin contact throughout device wear and operation. This is important for obtaining adequate ECG signal-to-noise-ratio. Electrode-skin contact can be indicated indirectly by measuring the impedance between adjacent electrodes. Normal electrode-electrode impedance is generally in the range of 1 to 15 k-ohms depending on the condition of the skin and the distance between the electrodes. Measurement and detection of electrode-electrode impedance can also be used to activate the patch device 10 automatically upon its placement on the abdomen skin. Automatic activation can also be accomplished during the removal of the patch device from its package, i.e. a pouch. For example, by incorporating open-circuit and/or short-circuit conditions between the electrodes within the package. These circuit conditions are altered during the removal of the patch device 10 from the package triggering the activation of the device. These and other automatic activation means and methods will be readily recognized by those skilled in the art of electronics and medical device packaging.

Although the invention is described herein with reference to the preferred embodiment, one skilled in the art will readily appreciate that other applications may be substituted for those set forth herein without departing from the spirit and scope of the present invention. Accordingly, the invention should only be limited by the claims included below.

1. A non-invasive integrated fetal monitor device for obtaining vital signs of a fetus, comprising:
at least one electrode for contacting the surface of the skin of a pregnant mother at or near her abdomen, said at least one electrode receiving a surface potential of a fetal ECG signal, wherein said fetal ECG signal is contaminated with a maternal ECG signal;
an amplifier for amplifying said ECG signals from said at least one electrode;
a processor for performing real-time processing and analysis of said amplified ECG signals;
a power source for powering said fetal monitor device;
a thin flexible substrate for housing said amplifier, said processor, said at least one electrode, and said power source; and
means for extracting filtered fetal ECG data, relatively free from maternal ECG components, from said amplified ECG signals.

2. The device of claim 1, said device having a form factor comprising:
a patch for adhesive attachment on the skin of said pregnant mother.

3. The device of claim 2, wherein said device has a thickness of less than 3.5 mm.

4. The device of claim 2, further comprising:
a flexible electronic circuit for interconnecting electronic components within to said at least one electrode.

5. The device of claim 1, further comprising:
at least one upper electrode positioned at an upper abdomen area or a chest area of said pregnant mother for obtaining a relatively pure maternal ECG signal.

6. The device of claim 5, said means for extracting filtered fetal ECG comprising:
means for subtracting said relatively pure maternal ECG signal obtained from said at least one upper electrode from said contaminated fetal ECG.

7. The device of claim 1, further comprising;
two or more abdominal electrodes positioned in a lower abdomen area of said pregnant mother.

8. The device of claim 1, further comprising:
means for automatically and dynamically selecting a network of electrodes for processing by said processor to yield optimal filtered fetal ECG signal according to a dynamic position of said fetus.

9. The device of claim 1, said means for extracting filtered fetal ECG comprising:
two or more abdominal electrodes positioned along an equal potential contour line orthogonal to a maternal cardiac vector; and
means for subtracting substantially similar maternal ECG data from unfiltered fetal ECG.

10. The device of claim 1, said means for extracting filtered fetal ECG comprising:
means for applying a singular value decomposition.

11. The device of claim 1, said means for extracting filtered fetal ECG comprising:
means for applying feature extraction of a fetal ECG R waveform.

12. The device of claim 1, further comprising:
an attachment means for allowing said device to be worn by said pregnant mother continuously for an extended period of time, including during her sleep.

13. The device of claim 12, wherein said extended period exceeds one week.

14. The device of claim 1, wherein said device is a disposable construct that is discarded after depletion of said power source.

15. The device of claim 1, wherein said device is waterproof.

16. The device of claim 1, wherein said processor comprises:
a digital signal processor.

17. The device of claim 1, further comprising:
a memory for storing ECG data.

18. The device of claim 1, further comprising:
means for receiving a wireless control signal from a remote control device.

19. The device of claim 18, said remote control device comprising:
a programming unit.

20. The device of claim 18, said remote control device comprising:
a magnet.

21. The device of claim 18, said means for receiving a wireless control signal comprising:
a sensor transducer incorporated in said device.

22. The device of claim 21, said sensor transducer comprising:
a miniature reed-switch.

23. The device of claim 21, said sensor transducer comprising:
an audio transducer.

24. The device of claim 1, further comprising:
means for sending stored vital sign data to a remote device.

25. The device of claim 24, said means for sending stored vital sign data comprising:
a trans-telephonic means for sending said data via a telephone.

26. The device of claim 1, further comprising:
a transducer for indicating vital signs.

27. The device of claim 26, said transducer comprising:
audible transducer means including a speaker and a buzzer.

28. The device of claim 26, said transducer comprising:
visual display means including a light emitting diode (LED) and a liquid crystal display (LCD).

29. The device of claim 26, said transducer comprising:
a vibrator for imparting tactile vibrations on the skin of said pregnant mother.

30. The device of claim 1, said power source comprising:
a battery.

31. The device of claim 1, further comprising:
a rechargeable power source; and
means for externally recharging said power source.

32. The device of claim 1, said substrate comprising:
a metal foil.

33. The device of claim 1, further comprising:
means for automatic powering and activation of said device upon either of opening of a package containing said device and placement of said device on the skin of said pregnant mother.

34. The device of claim 2, further comprising:
means for adhesively attaching said patch to the skin of said pregnant mother until said power source is depleted and said device ready for disposal.

35. The device of claim 2, further comprising:
means for reattachably adhering said patch to the skin of said pregnant mother.

36. The device of claim 1, further comprising:
means for detection and indication of multiple fetal ECGs.

37. A programmable, non-invasive fetal monitor patch for monitoring vital signs of a fetus, comprising:
at least one electrode for contacting the surface of the skin of a pregnant mother at or near her abdomen, said at least one electrode receiving a surface potential of a fetal ECG signal, wherein said fetal ECG signal is contaminated with a maternal ECG signal;
an ECG amplifier connected to said at least one electrode;
a processor;
a power source;
a wireless sensor element for receiving wireless commands from an external programming unit;
a thin, flexible substrate for housing said ECG amplifier, said processor, said at least one electrode, said power source, and said wireless sensor element;
means for extracting a filtered fetal ECG and for removing a maternal ECG component therefrom; and
means for configuring operation of said processor responsive to wireless commands received by said wireless sensor element from said external programming unit.

38. The fetal monitor patch of claim 37, said wireless sensor element comprising any of:
a reed-switch, coil, an RF receiver, an optical sensor, an audio transducer, and an ultrasonic transducer.

39. The fetal monitor patch of claim 37, said programming unit comprising:
a transmitting element comprising any of an electromagnet coil, an induction coil, an RF transmitter, an LED, an audio transducer, and an ultrasonic transducer.

40. The fetal monitor patch of claim 39, said programming unit comprising:
a housing for said transmitting element of said programming unit comprising a hand-held wand which is introduced in proximity to said fetal monitor during programming thereof.

41. A non-invasive, fetal monitor patch for monitoring vital signs of the fetus, comprising;
at least one electrode for contacting the surface of the skin of a pregnant mother at or near her abdomen, said at least one electrode receiving a surface potential of a fetal ECG signal, wherein said fetal ECG signal is contaminated with a maternal ECG signal;
an ECG amplifier connected to said at least one electrode;
a processor for receiving amplified ECG signals;
a power source for powering said fetal monitor device;
a flexible substrate for housing said ECG amplifier, said processor, said at least one electrode, and said power source;
means for extracting a filtered fetal ECG and for removing a maternal ECG component therefrom;
a memory for storing fetal ECG data therein; and
means for transmitting stored fetal ECG data from said memory to a remote instrument.

42. The fetal monitor patch of claim 41, said means for transmitting stored fetal ECG data comprising:
means for acoustically transmitting said stored fetal ECG data to a remote instrument via a telephone.

43. The fetal monitor patch of claim 41, said means for transmitting stored data to a remote instrument comprising:
means for transmitting said stored fetal ECG data to a remote instrument via the Internet.

44. A method of non-invasive fetal monitoring, comprising the steps of;
adhesively attaching a patch on the abdomen area of a pregnant woman, said patch comprising a thin flexible substrate, an ECG amplifier, a processor, at least one electrode contacting the skin of the pregnant woman in her abdomen area, a power source, and an alarm indicator;
amplifying a fetal ECG signal obtained from said at least one electrode, said fetal ECG signal being contaminated with a maternal ECG signal;
converting an amplified fetal ECG signal to fetal ECG data contaminated with maternal ECG data;
extracting filtered fetal ECG data from said amplified fetal ECG signal by removing said maternal ECG data therefrom;
computing at least one vital sign for said fetus from said filtered fetal ECG data; and
activating said alarm indicator automatically if said at least one vital sign is outside a predetermined limit.

45. The method of claim 44, said extracting step further comprising the step of:
removing relatively pure maternal ECG data obtained from at least one upper electrode placed on the upper abdomen or chest area of said pregnant woman.

46. The method of claim 44, said extracting step further comprising the step of:
removing maternal ECG data obtained from multiple abdominal electrodes.

47. The method of claim 46, further comprising the step of positioning said multiple abdominal electrodes in a substantially horizontal configuration.

48. A method of non-invasive fetal monitoring, comprising the steps of;
adhesively attaching a fetal monitor patch on an abdomen area of a pregnant woman, said patch incorporating within a thin flexible substrate, an ECG amplifier, an analog to digital converter, a processor, at least one electrode for contacting the skin of said pregnant woman in her abdomen area, a power source, and a heartbeat indicator;
amplifying a fetal ECG signal from said at least one electrode;
converting an amplified fetal ECG signal to fetal ECG data with said analog to digital converter, said fetal ECG data being contaminated with maternal ECG data;
extracting filtered fetal ECG data from said amplified fetal ECG signal by canceling maternal ECG data from contaminated fetal ECG data;
computing and obtaining at least one fetal vital sign from said filtered fetal ECG data; and
activating said heartbeat indicator with each incidence of a fetal QRS complex detected by said processor.

49. The method of claim 48, said heartbeat indicator comprising:
any of an audible transducer and a visual display device.

50. The method of claim 48, said heartbeat indicator comprising:
means for activation and deactivation of said heartbeat indicator.

51. The method claim 48, wherein said means for activation and deactivation comprise an external magnet brought in contact with or in proximity to said fetal monitor patch.
